(19) United States
(12) Patent Application Publication
(10) Pub. No.: US 2024/0309327 A1
(43) Pub. Date: Sep. 19, 2024

(54) CARDIOMYOCYTE PRODUCTION METHOD

(71) Applicants: MITSUBISHI CHEMICAL CORPORATION, Tokyo (JP); MYORIDGE CO. LTD., Kyoto-shi (JP)

(72) Inventors: Itsunari MINAMI, Kyoto (JP); Kazuma SHOJI, Kyoto (JP); Michiko YAMAZAKI, Tokyo (JP); Hiroshi KAWABATA, Tokyo (JP)

(73) Assignees: MITSUBISHI CHEMICAL CORPORATION, Tokyo (JP); MYORIDGE CO. LTD., Kyoto-shi (JP)

(21) Appl. No.: 18/411,134

(22) Filed: Jan. 12, 2024

Related U.S. Application Data
(63) Continuation-in-part of application No. PCT/JP2022/027420, filed on Jul. 12, 2022.

(30) Foreign Application Priority Data
Jul. 12, 2021 (JP) 2021-114913

(51) Int. Cl. C12N 5/077; C12N 5/00

(52) U.S. Cl. CPC C12N 5/0657; C12N 5/0037; C12N 5/0056; C12N 2501/998; C12N 2506/45

(57) ABSTRACT

The present invention relates to a method for producing a cardiomyocyte. In more detail, the present invention relates to a method for producing a cell population containing cardiomyocytes, including differentiation process from cells capable of differentiating into cardiomyocytes to cardiomyocytes, and the differentiation process includes culturing the cells in a serum-free medium containing α fetoprotein to obtain the cardiomyocytes.

### CROSS-REFERENCE TO RELATED APPLICATION

This application is based upon and claims the benefit of priority of the prior Japanese Patent Application No. 2021-114913 (filed on Jul. 12, 2021), the entire contents of which are incorporated herein by reference.

### TECHNICAL FIELD

### Field of the Invention

The present invention relates to a method for producing cardiomyocytes. More particularly, it relates to a method for producing a cell population containing mature cardiomyocytes at a high ratio from pluripotent stem cells.

### Background Art

As a treatment method to be employed instead of heart transplantation, transplantation of cardiomyocytes derived from stem cells has been desired to be realized. It is said that at least 10⁹ cells are necessary for transplanting cardiomyocytes to a human, and it is significant for realizing regenerative medicine for the heart to prepare a cell population containing mature and high-quality cardiomyocytes at a high purity. It is possible to concentrate, with a marker or the like, for increasing a purity, cardiomyocytes obtained by a known method, but a conventional cardiomyocyte induction method requires a large number of cytokines, and hence has a problem of high production cost.

The present inventors have developed methods for differentiating cardiomyocytes inexpensively and highly efficiently by utilizing a low molecular weight compound (Patent Literatures 1 to 3). These methods are protein-free differentiation methods in which a protein such as a cytokine is replaced with a low molecular weight compound. iPS cell-derived cardiomyocytes generally stay, however, in an immature stage similar to fetal cardiomyocytes, and take time to mature. As a countermeasure, a method for maturing cells by adding a specific small molecule has been also proposed, but only expression increase of endogenous TNNI3 gene is described, and it is not clear whether or not the quality or yield of cells is improved (Patent Literature 4).

α-fetoprotein is a component contained in fetal bovine serum, and is sometimes added to a medium as a serum replacement (Patent Literatures 5 to 8). It is known that α-fetoprotein promotes growth of stem cells as a serum replacement (Patent Literature 5) and is used for maintaining an undifferentiated state (Patent Literature 8), but a method for using it as a factor for inducing differentiation of cells into specific cells has not been known.

### CITATION LIST

### Patent Literature

Patent Literature 1: WO2012/026491
Patent Literature 2: WO2013/111875
Patent Literature 3: WO2015/182765
Patent Literature 4: WO2019/189554
Patent Literature 5: WO2008/148938 (JP2010-528642A)
Patent Literature 6: WO2011/125860
Patent Literature 7: WO2013/047763
Patent Literature 8: WO2011/067465 (JP2013-512667A)

### SUMMARY OF INVENTION

### Technical Problem

An object of the present invention is to provide a method for efficiently inducing mature cardiomyocytes in a short time.

### Solution to Problem

The present inventors have found that the yield of cardiomyocytes is improved, and a ratio of mature cardiomyocytes having developed cytoskeleton or atrial cardiomyocytes is increased by adding α-fetoprotein during differentiation into cardiomyocytes. The present invention is based on this finding, and provides the following [1] to [24]:

[1] A method for producing a cell population containing cardiomyocytes, comprising differentiation process from cells capable of differentiating into cardiomyocytes to cardiomyocytes, wherein the differentiation process comprises culturing the cells in a serum-free medium containing α fetoprotein to obtain the cardiomyocytes.

[2] The method according to [1], wherein the cells capable of differentiating into cardiomyocytes are pluripotent stem cells or mesodermal cells derived from pluripotent stem cells.

[3] The method according to [2], wherein the pluripotent stem cells are iPS cells.

[4] The method according to any one of [1] to [3], wherein the obtained cardiomyocytes are cardiomyocytes having a high expression level of hERG, KCNJ2, SCN5A, or MYL7.

[5] The method according to any one of [1] to [4], wherein the obtained cardiomyocytes are mature cardiomyocytes.

[6] The method according to any one of [1] to [5], wherein the cells are cultured in the serum-free medium containing α fetoprotein from start of the differentiation until beating of the cell is confirmed.

[7] The method according to any one of [1] to [6], wherein the cell is cultured in the serum-free medium containing α fetoprotein after beating of the cells is confirmed.

[8] The method according to any one of [1] to [7], wherein the obtained cell population comprises cardiomyocytes in a ratio of 80% or more, preferably 85% or more, and more preferably 90% or more when measured by flow cytometry.

[9] The method according to any one of [1] to [8], wherein the α fetoprotein is human α fetoprotein.

[10] The method according to any one of [1] to [9], wherein the serum-free medium contains the α fetoprotein in an amount of 5 to 100 μg/mL.

[11] The method according to any one of [1] to [10], wherein the serum-free medium is xeno-free and/or cytokine-free.

[12] The method according to any one of [1] to [10], wherein the serum-free medium is protein-free excluding α fetoprotein.

[13] A serum-free medium, comprising α fetoprotein, preferably 5 to 100 μg/mL of α fetoprotein, and a differentiation inducing factor for a cardiomyocyte.

[14] The medium according to [13], wherein the serum-free medium is xeno-free and/or cytokine-free.

[15] The medium according to or [14], wherein the serum-free medium is protein-free excluding α fetoprotein.

[16] The medium according to any one of to [15], wherein the differentiation inducing factor for cardiomyocytes contains any one of CHIR99021, prostratin, KY03-I, XAV939, A419259, and AG1478.

[17] The medium according to any one of to [16], wherein the differentiation inducing factor for cardiomyocytes is one or more selected from the group consisting of a Wnt signal activator (GSK3β inhibitor), a PKC activator, a Src inhibitor, and an EGF receptor inhibitor.

[18] A culture of a cell population containing cardiomyocytes, wherein the culture comprises α fetoprotein but does not comprise serum, wherein the cell population preferably contains cardiomyocytes in a ratio of 80% or more, preferably 85% or more, and more preferably 90% or more when measured by flow cytometry.

[19] A cardiomyocyte maturation promoter, comprising α fetoprotein as an active ingredient.

[20] A method for promoting maturation of cardiomyocytes, comprising adding α fetoprotein to a medium in a step of inducing differentiation into cardiomyocytes.

[21] A method for improving a yield of cardiomyocytes, comprising adding α fetoprotein to a medium in a step of inducing differentiation into cardiomyocytes. Preferably, the yield of the cardiomyocytes is improved, as compared with a case where α fetoprotein is not added, by at least 5%, more preferably 10% or more, 30% or more, or 50% or more.

[22] A method for improving a ratio of atrial cardiomyocytes, comprising adding α fetoprotein to a medium in a step of inducing differentiation into cardiomyocytes. Preferably, a ratio of MYL7-positive cells measured by FACS is 60% or more, or expression level of MYL7 gene measured by RT-PCR is improved, as compared with a case where α-fetoprotein is not added, by 50% or more, preferably 70% or more, 80% or more, 90% or more, and more preferably 100%. Alternatively, expression level of an ion channel is improved, as compared with that of immature cardiomyocytes, by 50% or more, preferably by 100%, more preferably by 200%, and further preferably by 400% or more.

[23] A method for improving a ratio of mature cardiomyocytes, comprising adding α fetoprotein to a medium in a step of inducing differentiation into cardiomyocytes. Preferably, a ratio of mature cardiomyocytes is improved, as compared with a case where α fetoprotein is not added, by at least 5% or more, more preferably 10% or more, or 20% or more.

[24] A pharmaceutical composition for use in treating heart diseases, comprising a cell population obtained by any one of the methods according to [1] to [12], and a pharmacologically acceptable carrier, wherein the cell population contains cardiomyocytes in a ratio of 80% or more, preferably 90% or more, and more preferably 95% or more when measured by flow cytometry, and is enriched with mature cardiomyocytes and/or atrial cardiomyocytes.

### Advantageous Effect of Invention

According to the present invention, mature cardiomyocytes can be efficiently induced in a short time. Since a serum-free medium is used in the method of the present invention, medium components can be more stably controlled, and a cell population containing mature cardiomyocytes suitable for clinical use at a high purity can be prepared inexpensively and in a short time. Besides, when the method of the present invention is combined with a PFCD method, pluripotent stem cells can be induced to cardiomyocytes in a xeno-free and cytokine-free manner.

### BRIEF DESCRIPTION OF DRAWINGS

FIG. 1A illustrates an AFP (50 μg/mL) addition period, and FIG. 1B illustrates a cardiomyocyte (cTnT positive cell) ratio obtained on day 19 after cultivation performed under respective conditions.

FIG. 2A illustrates an AFP (10 μg/mL) addition period, FIG. 2B illustrates a cardiomyocyte (cTnT positive cell) ratio obtained on day 28 after cultivation performed under respective conditions, and FIG. 2C illustrates MYL7 gene expression level, the ordinate of FIG. 2C indicating relative mRNA expression (that is, a relative mRNA expression level obtained assuming that the expression level of a control (A) is 1).

FIG. 3 illustrates an amount of phalloidin fluorescence obtained by adding AFP to cardiomyocytes to a final concentration of 10 μg/mL or 1 μg/mL, and culturing the resultant, the ordinate indicating a total amount of FITC fluorescence of phalloidin-FITC.

FIG. 4 illustrates an amount of phalloidin fluorescence obtained by adding, to cardiomyocytes, various AFP samples, and culturing the resultant, the ordinate indicating a total amount of FITC fluorescence of phalloidin-FITC.

FIGS. 5A-5C illustrate expression levels of α-MHC, β-MHC, and MYL7, FIGS. 5D-5F illustrate expression levels of hERG, that is, a voltage-dependent K⁺ channel marker, Kir2.1, Nav 1.5, that is, a voltage-dependent Na channel marker, and hHCN4, that is, a pacemaker specific ion channel, the ordinate of each graph indicating relative mRNA expression (that is, a relative mRNA expression level obtained assuming that the expression level of a control, measured under a control condition of N=3, is 1).

FIG. 6 illustrates phalloidin brightness (development of cytoskeleton) (FIG. 6A), cTnT brightness (development of myocardial fiber) (FIG. 6B), and the number of DAPI (number of cells) (FIG. 6C) obtained by adding 2% FBS, HSA, umbilical cord blood-derived AFP, HEK recombinant AFP, and umbilical cord blood-derived AFP+HSA.

### DESCRIPTION OF EMBODIMENTS

### 1. Method for Producing Cell Population Containing Cardiomyocytes

The present invention relates to a method for producing a cell population containing cardiomyocytes, including, in differentiation process from cells capable of differentiating into cardiomyocytes to cardiomyocytes, culturing the cells in a serum-free medium containing α-fetoprotein to obtain the cardiomyocytes.

In the present invention, the term “cardiomyocyte(s)” refers to a cell(s) constituting the myocardium, and is characterized by, for example, expression of cardiac troponin (cTnT) and beating. The cardiomyocytes have subtypes of a sinus node type, a ventricle type, and an atrial type. As described below, atrial cardiomyocytes are characterized by high expression of MYL7 gene or the like. Mature cardiomyocytes are also characterized by high expression of an ion channel.

In the present invention, the term “cells capable of differentiating into cardiomyocytes” are not especially limited as long as it is cells capable of differentiating into cardiomyocytes, and encompasses, for example, pluripotent stem cells, mesenchymal stem cells, myocardial progenitor cells, and mesodermal cells differentiated from pluripotent stem cells. The “cells capable of differentiating into cardiomyocytes” are preferably pluripotent stem cells, or mesodermal cells differentiated from pluripotent stem cells.

In the present invention, the term “pluripotent stem cell (s)” means a stem cell (s) having ability to differentiate into all the cell lineages of three germ layers (endoderm, mesoderm, and ectoderm). Examples of the pluripotent stem cell include an artificial pluripotent stem cell (iPS cell), an embryonic stem cell (ES cell), a sperm stem cell, and an embryonic germ cell.

Examples of the “iPS cell” include iPS cells established by introducing four factors of Oct3/4, Sox2, Klf4, and c-Myc into mouse or human cells (fibroblasts, peripheral blood cells, umbilical cord blood cells, and the like), and iPS cells established by introducing three factors of Oct3/4, Sox2, and Klf4 into mouse or human cells by Yamanaka, et al, of Kyoto University; iPS cells established by introducing four genes of OCT3/4, SOK2, NANOG, and LIN28 into mouse or human cells by Thomson, et al, of Wisconsin University, and iPS cells established by introducing six genes of OCT3/4, SOX2, KLF4, C-MYC, hTERT, and SV40 large T into mouse or human cells by Daley et al, of Harvard University. The iPS cell is preferably a clinical grade human iPS cell, and if necessary, cells having the same type of HLA as a target of transplantation are used.

The “ES cell” is not especially limited, and from an ethical point of view, an established ES cell line is preferably used. As the ES cell line, ES cell lines provided by RIKEN, Kyoto University, and NIH as well as ES cell lines commercially available from Cellartis can be used.

In the present invention, the term “myocardial progenitor cell (s)” means a progenitor cell (s) or a stem cell (s) having a tropism for differentiation into a cardiomyocyte. The myocardial progenitor cell (s) encompasses a c-kit-positive myocardial progenitor cell, and a c-kit-negative myocardial progenitor cell, and can be isolated from a heart tissue by a known method (WO2003/035838, WO2006/093276, and the like).

In the present invention, the term “mesodermal cells derived from pluripotent stem cells” refers to mesodermal cells differentiated from the above-described “pluripotent stem cell(s)”. The mesoderm refers to a cell population formed between the endoderm and the ectoderm at early development stage of an animal. Specifically, the “mesodermal cells derived from pluripotent stem cells” refers to a cell population obtained during induced differentiation of pluripotent stem cells into cardiomyocytes, and is characterized by expression of, for example, a mesodermal marker such as T, MIXL1, NODAL, MSX1, αSMA, NKX2.5, or αMHC. In the present invention, immature cardiomyocytes such as cardiomyocytes having an undeveloped cytoskeleton, cardiomyocytes having a low expression level of troponin T, troponin I, and βMHC, and cardiomyocytes having a high expression level of NKX2.5 are also encompassed in the “mesodermal cells derived from pluripotent stem cells”.

Cells used in the present invention are preferably cells of a mammal, and more preferably cells of a human.

In the present invention, the cells capable of differentiating into cardiomyocytes are cultured in a serum-free medium containing α-fetoprotein.

The “α-fetoprotein” is a glycoprotein produced in liver cells or yolk sac of an unborn baby. The “α-fetoprotein” is a marker for an endodermal early liver cells, and is known also as a tumor marker because it is produced in tumor cells such as liver tumor cells. The “α-fetoprotein” is abundantly contained in fetal bovine serum (FBS), and can be recombinant α fetoprotein, and is used as a serum replacement in some cases.

The α fetoprotein used in the present invention is preferably bovine α fetoprotein, or human α fetoprotein, and more preferably human α fetoprotein. Besides, in consideration of clinical use, the α-fetoprotein is preferably recombinant α fetoprotein, particularly recombinant human α fetoprotein.

The amount of the α fetoprotein to be added to the serum-free medium is appropriately set in accordance with the properties of cells to be used. The amount of the α fetoprotein is, for example, at least 1 μg/mL or more, 2 μg/mL or more, 3 μg/mL or more, preferably 5 μg/mL or more, and more preferably 10 μg/mL or more. The upper limit is preferably 500 μg/mL at most, more preferably 300 μg/mL, further preferably 200 μg/mL, particularly preferably 100 μg/mL, and still more preferably 50 μg/mL. The α fetoprotein is used in an amount of, for example, in a range of 1 to 200 μg/mL, preferably in a range of 5 to 100 μg/mL, and more preferably in a range of 10 to 50 μg/mL.

As the α fetoprotein used in the present invention, a composition containing another pharmacologically acceptable component in addition to the α fetoprotein may be used. The composition containing the α fetoprotein is not limited, and any of known reagents and the like can be used. Examples include BioVision AFP (Catalog No. P1585), LEE BIOSOLUTIONS AFP (Catalog No. 105-11), and HyTest AFP (Catalog No. 8F8), and in particular, HyTest AFP (Catalog No. 8F8) is preferred because toxicity of the solvent portion is low. Besides, when BioVision AFP or LEE BIOSOLUTIONS AFP is used, it is preferable to use it with another component contained in addition to AFP reduced or removed by buffer replacement.

The “serum-free medium” used in the present invention means a medium not containing an unadjusted or unpurified serum. The constitution of the serum-free medium will be described in detail below in “3. Medium”.

The cultivation of the cells in the serum-free medium containing the α fetoprotein is performed preferably at a stage where the cells have been differentiated into mesodermal cells, and preferably into immature cardiomyocytes. Immature cardiomyocytes are characterized, for example, during the differentiation, as cells on 0 to 7 days from start of the beating of cardiomyocytes, or by expression of a mesodermal marker such as T, MIXL1, NODAL, MSX1, αSMA, NKX2.5, or αMHC. A method employed when pluripotent stem cells are used as a starting material will be described in detail in the following section.

The “cardiomyocyte(s)” obtained by the method of the present invention is preferably a “mature cardiomyocyte(s)”. The term “mature cardiomyocyte(s)” means a cardiomyocyte(s) having developed cytoskeleton. The development of cytoskeleton can be confirmed by staining the actin filament of cells with, for example, phalloidin or the like. For example, when double staining with phalloidin and cTnT is performed by immunostaining, the amount (ratio) of the “mature cardiomyocyte(s)” having developed cytoskeleton can be determined.

The “mature cardiomyocyte(s)” is also characterized by a high expression level of voltage-dependent K⁺ channels (Kvl1.1 and Kir2.1) and a voltage-dependent Na channel (Nav1.5), which are known to increase in expression level when cardiomyocytes are matured. For example, when an RNA (mRNA) amount of hERG encoding Kvl1.1, KCNJ2 encoding Kir2.1, or SCN5A encoding Nav1.5 is measured by RT-PCR, the amount of mature cardiomyocytes as compared with that of a comparison target (such as a control) can be determined. It is noted that “high expression level” of an ion channel gene (hERG, KCNJ2, or SCN5A) means that the expression level is higher by 50% or more as compared with that of immature cardiomyocytes. The expression level of the ion channel gene is higher, as compared with that of immature cardiomyocytes, preferably by 100%, more preferably by 200%, and further preferably by 400% or more. Alternatively, when the number (ratio) of ion channel protein-positive cells is measured by FACS, the amount (ratio) of the “mature cardiomyocytes” can be more clearly determined. In this case, when the ratio of the ion channel-positive cells is 60% or more, it can be said that the cell population contains (mature) cardiomyocytes having a high expression level of the ion channel“.

The “cardiomyocytes” obtained by the method of the present invention are preferably “atrial cardiomyocytes”. The “atrial cardiomyocyte(s)” is characterized by a high expression level of MYL7, which is known to increase in expression level when atrial muscle is matured. For example, when the amount of MYL gene (mRNA) is measured by RT-PCR, the amount of the atrial cardiomyocytes as compared with a comparison target (such as a control) can be determined. It is noted that “high expression level” of MYL7 gene means that the expression level is higher by 50% or more as compared with that of immature cardiomyocytes. The expression level of MYL7 gene is higher, as compared with that of immature cardiomyocytes, preferably by 100%, more preferably by 200%, and further preferably 400% or more. Alternatively, when the number (ratio) of MYL protein-positive cells is measured by FACS, the amount (ratio) of the “atrial cardiomyocyte(s)” can be more clearly determined. In this case, when the ratio of the MYL7-positive cell is 60% or more, it can be said that the cell population contains (atrial) cardiomyocytes having a high expression level of MYL7”.

The “cell population containing cardiomyocytes” obtained by the method of the present invention is enriched in the above-described atrial cardiomyocytes.

The “cell population containing cardiomyocytes” obtained by the method of the present invention is enriched for the above-described mature cardiomyocytes.

Here, the term “enrichment” or “to be enriched” means that the ratio of the cells in the cell population is increased. For example, when the method of the present invention is employed, the atrial cardiomyocytes are enriched by 5%, 108, 20%, 30%, 40%, 50%, 60%, 70%, 80%, 858, 90%, 95%, 97%, 98%, or 99% as compared with that when it is not employed. Alternatively, when the method of the present invention is employed, the mature cardiomyocytes are enriched by 5%, 10%, 20%, 30%, 40%, 50%, 60%, 70%, 808, 85%, 90%, 95%, 97%, 988, or 99% as compared with that when it is not employed.

In general, for use in regenerative medicine for the heart, a cell population needs to contain cardiomyocytes in a ratio of at least 70%, and preferably 80% or more. Accordingly, the cell population containing cardiomyocytes at a high purity obtained by the method of the present invention is suitably used in regenerative medicine.

Herein, the “cell population containing cardiomyocytes” refers to a cell population containing the above-described cardiomyocytes. The cell population refers to a plurality of cells and is not limited in the shape. For example, it may be one where a plurality of cells are dispersed, or one where a plurality of cells are bonded to form a lump. Examples of the shape include a sheet, a lump, and a suspension containing the cells.

For identifying a cell, a “marker” specific to the cell can be used. The “marker” encompasses both a “marker protein” and a “marker gene”, and means a protein or a gene that is specifically expressed, or specifically deleted on a cell surface, in a cytoplasm, and/or in a nucleus or the like of a prescribed cell type. The marker protein is preferably a cell surface protein.

For detection of a marker protein such as cTnT, immunological assay using an antibody specific to the marker protein, such as ELISA, immunostaining, or flow cytometry, can be used. Detection of a marker gene such as MYL7 can be performed by utilizing, for example, RT-PCR, a microarray, a biochip or the like.

Herein, the term “to be expressed” or “positive expression” means that a protein or a gene is expressed in an amount detectable by any of methods known in this field (or in an amount higher than the background intensity). Herein, the term “not to be expressed” or “negative expression” means that the expression level of a protein or a gene is equal to or lower than the detection limit by all or any one of the known methods.

2. Production of Cardiomyocyte from Pluripotent Stem Cell

The present invention provides a method for producing a cell population containing cardiomyocytes, including adding α fetoprotein in differentiation process from pluripotent stem cells to cardiomyocytes.

In the present invention, the term “differentiation process” means process from starting time point of differentiation. The starting time point of differentiation specifically means a time point of change to a medium for differentiation. It is, for example, a time point when a medium for maintaining an undifferentiated state of cells capable of differentiating into cardiomyocytes is changed to a medium for differentiation. An end time point of the “differentiation process” is not limited, and is preferably up to a stage where the cells are sufficiently matured, for example, matured similarly to adult cardiomyocytes. Accordingly, the “differentiation process” includes a maturing step (described below) for further maturing cardiomyocytes characterized by expression of cardiac troponin (cTnT) or beating.

The number of times of adding the α fetoprotein is not limited, and is appropriately set in accordance with a method to be employed, and properties of a desired cell population of cardiomyocytes.

### 2.1 Application to Known Myocardial Differentiation Method

Specifically, the method of the present invention includes:
1) a step of differentiating pluripotent stem cells to cardiomyocytes in a medium containing α fetoprotein; and
2) a step of culturing the differentiated cells in a serum-free medium containing α fetoprotein to obtain cardiomyocytes.

(Here, a medium not containing α fetoprotein means that α fetoprotein is contained in an amount not exhibiting its function, and a case where a very small amount of α fetoprotein of, for example, less than 1 μg/mL is contained is not excluded.)

The induced differentiation of pluripotent stem cells into cardiomyocytes can be conducted by adding, to the medium, a differentiation inducing factor for cardiomyocytes. The differentiation inducing factor is not limited, and any of factors known in this field can be used. As the differentiation inducing factor for cardiomyocytes, for example, a cytokine is used. Examples of the cytokine include Activin A, BMP4, FGF2, DKK1, and VEGF. Alternatively, as a method not using a cytokine, for example, a Wnt signal activator (GSK3β inhibitor), a PKC activator, a Wnt signal inhibitor, a Src inhibitor, or an EGF receptor inhibitor can be used, and such a method is described in literatures describing a PFCD method described below. The differentiation can be conducted by using such a factor in accordance with a known method, and examples of the method include a method of J. Zhang et al., (https://www.ncbi.nlm.nih.gov/pmc/articles/PMC3482164/), methods described in WO2005/033298, WO2007/002136, WO2007/126077, WO2009/017254, WO2009/118928, US20200407687A1, U.S. Pat. No. 9,453,201 and the like, and the PFCD method developed by the present inventors described below.

The timing of adding the α fetoprotein differs depending on a method to be employed, and properties of a desired cell population of cardiomyocytes, and is not limited as long as it is after starting the differentiation. For example, a method in which it is added at an early stage of the differentiation process (method 1), a method in which it is added at a late stage of the differentiation process (method 2), or a method in which it is added both at an early stage and a late stage of the differentiation process (method 3) can be employed. From the viewpoint of the yield of mature cardiomyocytes, it is preferable to add AFP without limiting a period at an early stage of the differentiation process.

### Method 1:

1) Cells are cultured in a medium not containing α fetoprotein from the start of the differentiation until beating of the cells is confirmed, and 2) after confirming the beating of the cells, the cells are cultured in a serum-free medium containing the α fetoprotein. Specifically, for about 3 days after starting the differentiation, cells are cultured in a medium not containing α fetoprotein, and thereafter, the resultant cells are cultured in a serum-free medium containing the α fetoprotein for at least 3 days or more, and preferably 4 days or more.

### Method 2:

1) Cells are cultured in a serum-free medium containing the α fetoprotein from the start of the differentiation until beating of the cells is confirmed, and 2) after confirming the beating of the cells, the cells are cultured in a medium not containing α fetoprotein. Specifically, for about 3 days after starting the differentiation, cells are cultured in a serum-free medium containing the α fetoprotein, and thereafter, the resultant cells are cultured in a medium not containing α fetoprotein for at least 3 days or more, and preferably 4 days or more.

### Method 3:

1) Cells are cultured in a medium not containing α fetoprotein from the start of the differentiation until beating of the cells is confirmed, and 2) also after confirming the beating of the cell, the cells are cultured in a serum-free medium containing the α fetoprotein. Specifically, for about 3 days after starting the differentiation, cells are cultured in a serum-free medium containing the α fetoprotein, and thereafter, the resultant cells are cultured in a serum-free medium containing the α fetoprotein for at least 3 days or more, and preferably 4 days or more.

### 2.2 Application to PFCD Method

The present inventors have developed a method for inducing cardiomyocytes from pluripotent stem cells using a low molecular weight compound (PFCD method), and this method can be suitably employed in the present invention (WO2012/026491, WO2013/111875, WO2015/182765, US2013/0183753, and US2014/0127807). Now, application of the method of the present invention to the PFCD method will be specifically described.

### [Maintenance/Growth of Pluripotent Stem Cell]

Pluripotent stem cells may be, prior to the differentiation process, cultured in an appropriate medium to be acclimated/maintained, and if necessary, expanded (grown). As the medium used for such acclimation/maintenance/expansion, commercially available media such as mTeSR™ 1, mTeSR™ 2, StemFit®, CTS Knock Out SR Xeno Free, Essential 8, CTS Essential 8, hESF9, CDM, STEMPRO, DMEM, IMDM, RMPI, DMEM/F12, and αMEM can be used.

### [Differentiation Step]

In accordance with the PFCD method, induced differentiation of pluripotent stem cells into cardiomyocytes is conducted. The number of days of the differentiation step is at least 4 days or more, preferably 5 days or more, more preferably 4 to 7 days, most preferably 4 to 6 days, and for example, 5 or 6 days.

The differentiation step is divided into two steps: a step (1) of culturing pluripotent stem cells in a medium containing a Wnt signal activator (GSK3β inhibitor) and a PKC activator, and a step (2) of culturing the cells obtained in the step (1) in a medium containing a Wnt signal inhibitor, a Src inhibitor, and an EGF receptor inhibitor.

As the “Wnt signal activator”, for example, GSK3β inhibitors such as BIO, CHIR99021, CHIR98014, TDZD-8, SB216763, TWS-119, 1-azakenpaullone, SB216763, SB415286, AR-A0144-18, CT99021, CT20026, and TWS119 can be used. The Wnt signal activator is preferably BIO or CHIR99021, and more preferably CHIR99021.

Examples of the “PKC activator” include Phorbol 12-myristate 13-acetate (PMA), prostratin, Bryostatin 1, Bryostatin 2, FR236924, (-)-Indolactam V, PEP005, Phorbol 12,13-dibutyrate, SC-9, SC-10, 1-Oleoyl-2-acetyl-sn-glycerol, 1-O-Hexadecyl-2-O-arachidonyl-sn-glycerol, 1-O-Hexadecyl-2-O-arachidonyl-sn-glycerol, 1,2-Dioctanoyl-sn-glycerol, PIP2, Resiniferatoxin, Phorbol 12,13-Dihexanoate, Mezerein, Ingenol 3-Angelate, RHC-80267, DCP-LA, and Lipoxin A4. The PKC activator is preferably PMA or prostratin, and more preferably prostratin.

Examples of the “Wnt signal inhibitor” include IWP2, IWP4, XAV939, IWR1, and compounds described in WO2012/026491: KY02111 KY010104, T61164, KY02114, KY01045, KY01040, KY02109, KY010104, KY01043, KY01046, PB2852, N11474, PB2572, PB2570, KY02104, SO087, SO102, SO096, SO094, SO3031 (KY01-I), SO2031 (KY02-I), SO3042 (KY03-I), and SO2077. The Wnt signal inhibitor is preferably XAV939, or the compounds described in WO2012/026491.

Examples of the “Src inhibitor” include A419259, SU6656, PP1, 1-Naphthyl PP1, PP2, Indirubin-3′-(2,3-dihydroxypropyl)-oximether, TX-1123, Src Kinase Inhibitor I (CAS 179248-59-0), AZM475271, Bosutinib, Herbimycin A, KB SRC 4, MNS, PD166285, and TC-S7003. The Src inhibitor is preferably A419259 or SU6656, and more preferably A419259.

Examples of the “EGF receptor inhibitor” include AG1478, gefitinib, afatinib, ARRY334543, AST1306, AZD8931, BIBU1361, BIBX1382, BPDQ, BPIQ-I, BPIQ-II, canertinib, CL-387, 785, CUDC101, dacomitinib, vandetanib, EGFR inhibitor III (N-(4-((3,4-dichloro-6-fluorophenyl)amino)-quinazoline-6-yl)-2-chloroacetamide, CAS 733009-42-2), EGFR/ErbB-2 inhibitor (4-(4-benzyloxyanilino)-6,7-dimethoxyquinazoline, CAS 179248-61-4), erlotinib, GW583340, GW2974, HDS029, lapatinib, WHI-P154, OSI-420, PD153035, PD168393, PD174265, pelitinib, Compound 56, XL657, PP3, AG-490, AG555, tyrphostin B42, tyrphostin B44, AG556, AG494, AG825, RG-13022, DAPH, EGER Inhibitor (cyclopropanecarboxylic acid (3-(6-(3-trifluoromethyl-phenylamino)-pyrimidin-4-ylamino)-phenyl)-amide, CAS 879127-07-8), erbstatin analog (methyl 2,5-dihydroxycinnamate, CAS 63177-57-1), JNJ28871063, tyrphostin 47, lavendustin A, lavendustin C, lavendustin C methylate, LFM-A12, TAK165, TAK285, tyrphostin 51, tyrphostin AG183, tyrphostin AG528, tyrphostin AG99, tyrphostin RG14620, WZ3146, WZ4002, WZ8040, butein, and tyrphostin AG112. The EGF receptor inhibitor is preferably AG1478 or gefitinib, and more preferably AG1478.

One each of the Wnt signal activators (GSK3β inhibitors), the PKC activators, the Wnt signal inhibitors, the Src inhibitors, and the EGF receptor inhibitors described above may be used, or a combination of two or more each of these may be used.

In the method of the present invention, a period of the step (1) and the step (2), and a period from the end of the step (1) to the start of the step (2) can be appropriately changed in accordance with the type of the cell and the like. The step (2) may be started immediately after the end of the step (1), or may be started after a prescribed time period from the end of the step (1). For example, after the end of the step (1), the resultant cells may be cultured for 1 to 2 days, preferably 1 day in a medium containing none of a Wnt signal activator, a PKC activator, a Wnt signal inhibitor, a Src inhibitor, and an EGF receptor inhibitor, and then the step (2) may be started with the medium changed to a medium containing a Wnt signal inhibitor, a Src inhibitor, and an EGF receptor inhibitor.

A culture period of the step (1) is appropriately set in accordance with the cells to be used, and for example, the step (1) is conducted for 1 to 3 days, and subsequently, the step (2) is conducted, immediately after the end of the step (1) or 1 to 2 days after the end of the step (1), for 2 to 13 days, preferably 3 to 10 days, more preferably 4 to 10 days, and further preferably 4 to 8 days.

Since the step (1) corresponds to a first stage of myocardial differentiation that is a differentiation period from pluripotent stem cells to mesoderm, the period of the step (1) can be determined based on expression of a mesoderm-related gene (s). Examples of the mesoderm-related gene include T, MIXL1, and NODAL. The step (2) corresponds to a second stage of the myocardial differentiation of differentiation of mesoderm into cardiomyocytes, and hence the period can be determined by confirming differentiation into cardiomyocytes. The differentiation into cardiomyocytes can be confirmed based on the number of beating cardiomyocytes, expression of a myocardial marker (s), expression of an ion channel (s), a reaction (s) to electrophysiological stimulation, or the like. Examples of the myocardial marker include αMHC, βMHC, CTnT, α actinin, and NKX2.5. Examples of the ion channel include HCN4, Nav1.5, Cav1.2, Cav3.2, hERG, and KCNQ1.

The medium used in the differentiation step is also preferably a serum-free medium, and any of basal media as those described in “3. Medium for Maturing Cardiomyocyte” can be used, and the medium may be supplemented with various vitamins, amino acids, and the like in addition to the above-described low molecular weight compound. Besides, the cultivation is performed preferably as suspension cultivation using a low-adhesion container (dish). In addition, the details of the cultivation conditions accord with those described in WO2015/182765.

Cells obtained in the differentiation step are basically immature cardiomyocytes, and hence although a cardiomyocyte marker is expressed, development of the cytoskeleton and expression of an atrial cardiomyocyte marker MYL7, which are characteristics of a mature cardiomyocyte, are insufficient.

### [Maturing Step]

In a maturing step, the cells obtained in the differentiation step are cultured in a serum-free medium containing the α fetoprotein in accordance with the method described in “1. Method for Producing Cell Population Containing Cardiomyocytes”. The medium used in the maturing step does not contain the low molecular weight compound used in the differentiation step.

A culture period in the medium containing the α fetoprotein is not especially limited as long as it is after starting the differentiation. As described above, the α fetoprotein may be added at an early stage of the differentiation process, or may be added at a late stage. In other words, it may be added in the step 1, may be added in the step 2, or may be added in both the steps. The timing of adding the α fetoprotein may be determined based on expression of a myocardial marker. Specifically, the cultivation in the medium containing the α fetoprotein may be conducted after starting the differentiation until start of expression of a myocardial marker, or may be started at a timing when a myocardial marker starts to express. Even though a myocardial marker starts to express, the myocardial differentiation is not completed at this point, but the differentiation slowly progresses over about 1 to 2 weeks from this point. Therefore, although some cells start to express a myocardial marker, the other most cells are still at a stage of mesodermal cells, and hence, when the cells are cultured in the medium containing the α fetoprotein at this timing, the differentiation into the cardiomyocytes is accelerated.

There is no large difference in the yield of cardiomyocytes between a case where the cultivation in the medium containing the α fetoprotein is conducted at an early stage of the differentiation process (first stage of the differentiation cultivation), for example, within about 1 to 6 days after the end of the differentiation cultivation, and a case where it is conducted at a late stage of the differentiation process (second stage of the differentiation cultivation), for example, within about 7 to 18 days after the end of the differentiation cultivation, but the ratio of atrial cardiomyocytes is larger in the latter case.

In the maturing step, the cultivation is continued with the medium appropriately exchanged until a desired amount of cardiomyocytes, or a desired amount of mature cardiomyocytes or atrial cardiomyocytes are obtained. In general, the number of days of the step (2) is 1 day or more, preferably 3 days or more, and more preferably 5 days or more. For example, it is 2 to 4 days, preferably 4 to 7 days, and more preferably 7 to 14 days.

### 3. Medium for Maturing Cardiomyocyte

The present invention also provides a medium for cardiomyocyte differentiation. The medium of the present invention contains 5 to 100 μg/mL of α fetoprotein, does not contain serum, and contains a differentiation inducing factor for cardiomyocytes. The differentiation inducing factor for cardiomyocytes is the same as that described above.

The term “serum-free medium” means a medium not containing an unadjusted or unpurified serum, and a medium in which a purified blood-derived component or animal tissue-derived component (such as a growth factor) is mixed corresponds to a serum-free medium.

As a basal medium, any of media usable for culturing animal cells, such as DMEM medium, BME medium, αMEM medium, serum-free DMEM/F12 medium, BGJb medium, CMRL1066 medium, Glasgow MEM medium, Improved MEM Zinc Option medium, IMDM medium, Medium 199 medium, Eagle MEM medium, Ham's medium, RPMI 1640 medium, Fischer's medium, McCoy's medium, and Williams E medium, can be used, and stem cell media such as KnockOut™ DMEM, Medium 154, StemPro® hESC SFM, Essential 8, and Stemfit can be also used.

The medium may contain a “serum replacement”. Examples of the serum replacement include albumin (such as lipid-rich albumin), transferrin, a fatty acid, a collagen precursor, a trace element (such as zinc, or selenium), B-27® supplement, N2 supplement, knockout serum replacement (KSR: Invitrogen), 2-mercaptoethanol, and 3′ thiolglycerol. It is noted that the “serum replacement” used herein does not encompass α fetoprotein.

The medium may be appropriately supplemented with various nutrient sources necessary for maintaining and growing cells, and various components necessary for differentiation. For example, as the nutrient sources, carbon sources such as glycerol, glucose, fructose, sucrose, lactose, honey, starch, and dextrin, hydrocarbons such as a fatty acid, fats and oils, lecithin, and alcohols, nitrogen sources such as ammonium sulfate, ammonium nitrate, ammonium chloride, urea, and sodium nitrate, inorganic salts such as common salt, potassium salt, phosphoric acid salt, magnesium salt, calcium salt, iron salt, and manganese salt, monopotassium phosphate, dipotassium phosphate, magnesium sulfate, sodium chloride, ferrous sulfate, sodium molybdate, sodium tungstate, and manganese sulfate, various vitamins, and amino acids can be contained.

The serum-free medium is preferably xeno-free and/or cytokine-free. In one embodiment, the serum-free medium is protein-free, containing no proteinaceous component (such as albumin) in addition to the α fetoprotein.

The α fetoprotein is preferably bovine α fetoprotein or human α fetoprotein, and more preferably human α fetoprotein. Besides, the α fetoprotein is preferably recombinant α fetoprotein, particularly preferably recombinant human α fetoprotein.

The amount of the α fetoprotein added to the serum-free medium is 5 to 100 μg/mL. The lower limit is preferably 6 μg/mL, 7 μg/mL, 8 μg/mL, or 9 μg/mL, and more preferably 10 μg/mL. The upper limit is preferably 90 μg/mL, 80 μg/mL, 70 μg/mL, or 60 μg/mL, and more preferably 50 μg/mL. The amount is more preferably in a range of 10 to 50 μg/mL.

### 4. Cardiomyocyte Maturation Promoter

The present invention also provides a cardiomyocyte maturation promoter containing α fetoprotein as an active ingredient. The cardiomyocyte maturation promoter of the present invention may contain, in addition to the α fetoprotein, a carrier acceptable for cell cultivation.

Herein, the term “promotion” or “to promote” means that a result higher than that of a comparison target such as a control is obtained. It means that a result, for example, 1.1-fold, 1.2-fold, 1.5-fold, 2-fold, 3-fold, 4-fold, 5-fold, 6-fold, 7-fold, 8-fold, 9-fold, 10-fold, or further higher as compared with that of a comparative target is obtained, and it is preferable to obtain a statistically significantly high result.

### 5. Pharmaceutical Composition or Cardiac Cell Sheet for Treating Heart Disease

According to the method of the present invention, a cell population containing cardiomyocytes at a high ratio is obtained. In particular, a cell population containing mature cardiomyocytes or MYL7-positive atrial cardiomyocytes at a high ratio is obtained. For example, the cell population obtained by the method of the present invention contains cardiomyocytes in a ratio, when measured by flow cytometry, of 80% or more, preferably 85% or more, and further preferably 90% or more, for example, 91%, 92%, 93%, or 94% or more, and still further preferably 95% or more. The cell population of the present invention containing cardiomyocytes, particularly mature or atrial cardiomyocytes at a high ratio is useful for treatment of a heart disease, and the present invention provides a pharmaceutical composition or cardiac cell sheet for treating such a heart disease, and a method for producing the same.

For example, the cell population containing cardiomyocytes obtained by the method of the present invention can be used to prepare a pharmaceutical composition to be administered (injected) into a diseased part together with a pharmacologically acceptable carrier.

Examples of the pharmacologically acceptable carrier include sterile water, saline, a medium (particularly, a medium used for culturing mammal cells such as RPMI), and a biological buffer such as PBS. To the pharmaceutical composition, a vegetable oil, an emulsifier, a suspension, a surfactant, a stabilizer, an excipient, a preservative, or a binder may be added if necessary.

Examples of an aqueous solution for injection include saline, a medium, a biological buffer such as PBS, an isotonic solution containing glucose or another auxiliary agent, for example, D-sorbitol, D-mannose, D-mannitol, and sodium chloride, and these may be used together with an appropriate dissolution assisting agent, such as alcohol, specifically, ethanol, polyalcohol, propylene glycol, polyethylene glycol, a nonionic surfactant, for example, polysorbate 80, HCO-50 or the like.

Alternatively, the cell population containing cardiomyocytes, or the cardiomyocytes obtained by the method of the present invention may be layered to be processed into a sheet shape, and thus, a cardiac cell sheet can be prepared.

Examples of the target heart disease include ischemic heart diseases such as myocardial infarction and angina, and cardiac arrest.

### 7. Culture of Cell Population Containing Cardiomyocytes

The present invention also provides a cell population containing cardiomyocytes. The culture contains α-fetoprotein but does not contain serum. The cell population in the culture is enriched with cardiomyocytes, preferably mature cardiomyocytes, and contains cardiomyocytes, preferably mature cardiomyocytes in a ratio, when measured by flow cytometry, of 80% or more, preferably 85% or more, and further preferably 90% or more. Therefore, the culture of the present invention can be suitably used in regenerative medicine.

### 8. Miscellaneous

The present invention also provides a method for promoting maturation of cardiomyocytes by adding α-fetoprotein to a medium in a step of inducing differentiation into cardiomyocytes.

Besides, the present invention also provides a method for improving the yield of cardiomyocytes by adding α-fetoprotein to a medium in a step of inducing differentiation into cardiomyocytes. The yield of cardiomyocytes is preferably improved, as compared with a case where-fetoprotein is not added, by at least 5%, more preferably 10% or more, 30% or more, or 50% or more.

The present invention also provides a method for improving a ratio of atrial cardiomyocytes by adding α-fetoprotein to a medium in a step of inducing differentiation into cardiomyocytes. Preferably, a ratio of MYL7-positive cells measured by FACS is 60% or more, or expression level of MYL7 gene measured by RT-PCR is improved, as compared with a case where α-fetoprotein is not added, by 50% or more, and more preferably 100% or more.

The present invention also provides a method for improving a ratio of mature cardiomyocytes by adding α-fetoprotein to a medium in a step of inducing differentiation into cardiomyocytes. Preferably, the ratio of mature cardiomyocytes is improved, as compared with a case where α-fetoprotein is not added, by at least 5% or more, more preferably 10% or more, or 20% or more.

### EXAMPLES

Now, the present invention will be specifically described with reference to examples, and it is noted that the present invention is not limited to these examples.

### Example 1

Method (Addition of 50 μg/mL AFP)

Human iPS cells (253G1 strain) were adherent cultured in Essential 8 medium in a 10 cm dish (Falcon, 353003) coated with iMatrix-511 (Matrixome). When 80 to 90% confluent, the cells were peeled off with EDTA, seeded at about 1×10⁶ cells/well into a low adhesion 6-well plate (Corning, 3471), and cultured for 2 days to form an iPS cell spheroid.

A method of inducing differentiation into cardiomyocytes was conducted basically according to a method described in WO2015/182765. Specifically, in a medium of IMDM:DMEM of 1:1 containing an antibiotic and an amino acid (Table 1), 2 μM CHIR99021 and 1 μM prostratin were added to perform suspension cultivation for 2 days in a low-adhesion dish. On day 3, the medium was changed to a medium supplement with 4 μM KY03-1, 2 μM XAV939, 0.3 μM A419259, and 8 μM AG1478 (Table 1), followed by suspension cultivation until day 6.

TABLE 1

| Formulation | Amount | |
|---|---|---|
| IMDM | 242 | mL |
| DMEM | 242 | mL |
| MEM non-essential amino acid | 5 | mL |
| Penicillin-Streptomycin | 5 | mL |
| 0.2M L-glutamine | 5 | mL |
| 1M L-carnitine | 100 | μL |
| Ascorbic acid | 50 | mg |
| 0.5M creatine | 1 | mL |

On and after day 6, suspension cultivation was conducted in a medium not containing a low molecular weight compound (Table 1) in the presence or absence of AFP under each of the following conditions A to C (n=3 for each). As a composition containing AFP, human umbilical cord blood-derived AFP (BioVision, P1585-1000) was used.
(A) AFP not added (control)
(B) AFP added to a final concentration of 50 μg/mL in a period after starting differentiation from day 0 to day 6
(C) AFP added to a final concentration of 50 μg/mL in a period from day 7 to day 13

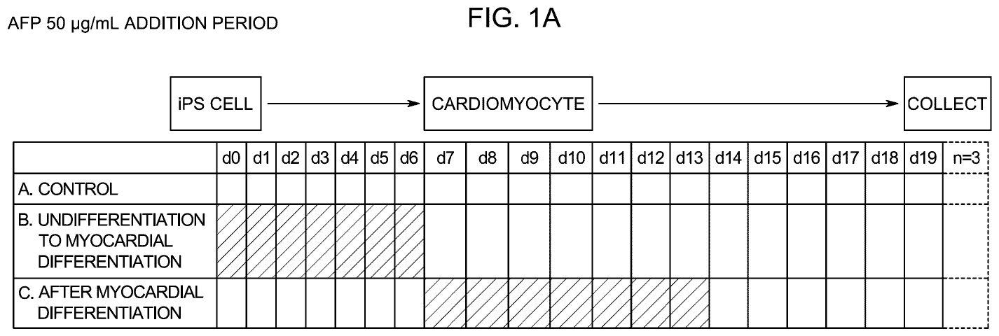

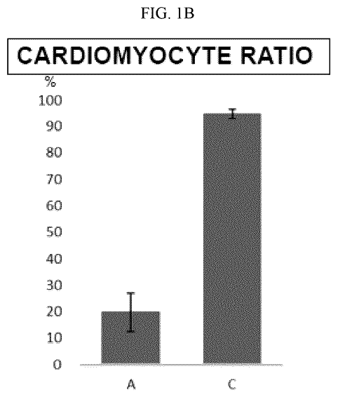

The cells were collected on day 19, subjected to single cell suspension by treatment with trypsin, and the number of cells was counted. Thereafter, a cardiomyocyte ratio (ratio of cTnT-positive cells) was checked by FACS analysis. In the FACS, Accuri C6 Plus (BD) was used. In FIG. 1A, “d0”, “d1”, etc. means day 0, day 1, etc. after starting the differentiation. Colored cells of “d3”, “d5” and the like indicate days of medium exchange.

### Results

Under the condition B (AFP added from day 0 to day 6), cell death was caused, and the analysis could not be performed. Under the condition C (AFP added from day 7 to day 13), the cTnT positive-cell ratio was high as compared with that of the control, and myocardial purity was improved (Table 2, FIG. 1).

TABLE 2

| | | Number of Cells After Single Cell | | | |
|---|---|---|---|---|---|
| | | Suspension | | | cTnT |
| | | | | Living Cell Ratio | Positive |
| | | Total(×10e5) | Live(×10e5) | (%) | Ratio (%) |
| A. Control | A-1 | 22.5 | 8.01 | 35.6 | 18.2 |
| | A-2 | 7.08 | 3.46 | 48.9 | 11.8 |
| | A-3 | 6.62 | 3.21 | 48.5 | 29.4 |
| B. | B-1 | — | — | — | — |
| Undifferentiation | B-2 | — | — | — | — |
| to Myocardial | B-3 | — | — | — | — |
| Differentiation | | | | | |
| C. After | C-1 | 18.2 | 5.84 | 32.1 | 93.6 |
| Myocardial | C-2 | 19.8 | 7.58 | 38.3 | 96.1 |
| Differentiation | C-3 | 19.9 | 4.79 | 24.1 | 91.8 |

### Example 2

Method (Addition of 10 μg/mL AFP)

Human iPS cells (253G1 strain) were adherent cultured in Essential 8 medium in a 10 cm dish (Falcon, 353003) coated with iMatrix-511 (Matrixome). When 80 to 90% confluent, the cells were peeled off with EDTA, seeded at about 1×10⁶ cells/well into a low adhesion 6-well plate (Corning, 3471), and cultured for 2 days to form an iPS cell spheroid.

A method of inducing differentiation into cardiomyocytes was conducted basically according to a method described in WO2015/182765 (see Example 1). In the same manner as in Example 1, differentiation of iPS cells was conducted under the following conditions A to D (n=3 for each). As a composition containing α-fetoprotein (AFP), human umbilical cord blood-derived AFP (BioVision, P1585-1000) was used.
(A) AFP not added (control)
(B) AFP added to a final concentration of 10 μg/mL in a period after starting differentiation from day 0 to day 6
(C) AFP added to a final concentration of 10 μg/mL in a period from day 7 to day 12
(D) AFP added to a final concentration of 10 μg/mL in a period from day 13 to day 18

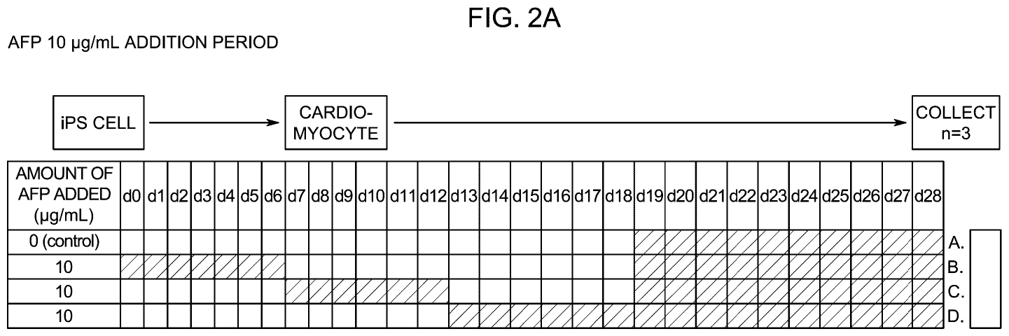

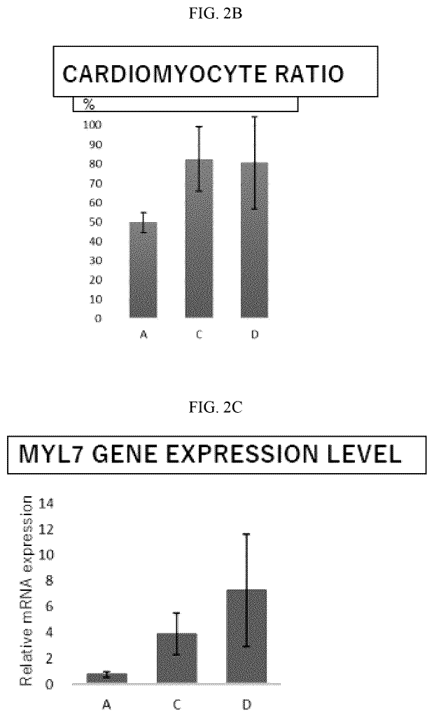

The cells were collected on day 28, and from a half amount of the collected cells, RNA was collected with miRNeasy Mini Kit (Qiagen K. K.) to analyze the expression level of Human myosin light chain 7 (MYL7), that is, an atrial muscle marker gene, by quantitative PCR (PowerUp SYBR Green Master Mix, Thermo Fisher Scientific) with TATA-Box Binding Protein (TBP) gene used as internal standard. The other half amount of the cells was subjected to single cell suspension by treatment with trypsin, and the number of cells was counted. Thereafter, a cardiomyocyte ratio (ratio of cTnT-positive cells) was checked by FACS analysis. In the FACS, Accuri C6 Plus (BD) was used. In FIG. 2A, “d0”, “d1”, etc. means day 0, day 1, etc. after starting the differentiation. Colored cells of “d3”, “d5” and the like indicate days of medium exchange.

### Results

When the addition concentration was 10 μg/mL, cell death was caused, and the differentiation could not be evaluated under the condition B (added from day 0 to day 6) in the same manner as in the case where the addition concentration was 50 μg/ML. Under the condition C (added from day 7 to day 12) and the condition D (added from day 13 to day 18), the cTnT positive-cell ratio was high as compared with that of the control, and the myocardial purity was improved (Table 3, FIG. 2B). Besides, MYL7 of the atrial muscle marker was increased more remarkably than the improvement trend of the myocardial purity under the conditions C and D of the AFP addition (FIG. 2C). The cells were probably differentiated into atrial cardiomyocytes.

TABLE 3

| | | Number of Cells After | | | | cTnT |
|---|---|---|---|---|---|---|
| | | Single Cell Suspension | | | cTnT | Positive |
| | | Total | Live | Living Cell | Positive | Ratio |
| | | (×10e5) | (×10e5) | Ratio (%) | Ratio (%) | (NC, %) |
| A. Control | A-1 | 99.6 | 61.9 | 62.2 | 53.4 | 0.2 |
| | A-2 | 74.9 | 45.9 | 61.3 | 53.5 | 0.2 |
| | A-3 | 68.3 | 48.3 | 70.7 | 42.4 | 0.1 |
| B. | B-1 | — | — | — | — | — |
| Undifferentiation | B-2 | 34.3 | 30.1 | 87.7 | 0.3 | 0.0 |
| to Myocardial | B-3 | — | — | — | — | — |
| Differentiation | | | | | | |
| C. After | C-1 | 65.4 | 43.4 | 66.4 | 94.6 | 0.1 |
| Myocardial | C-2 | 54.2 | 37.9 | 70.0 | 59.0 | 0.0 |
| Differentiation | C-3 | 54.4 | 32.7 | 60.1 | 94.2 | 0.0 |
| (first stage) | | | | | | |
| D. After | D-1 | 25.4 | 11.2 | 44.0 | 97.2 | 0.2 |
| Myocardial | D-2 | 18.7 | 14.1 | 75.1 | 98.4 | 0.4 |
| Differentiation | D-3 | 36.7 | 13.6 | 37.0 | 46.9 | 0.0 |
| (second stage) | | | | | | |

NC: Negative Control

### Example 3

Frozen cardiomyocytes (Myoridge Co., Ltd., H-011106: 30 days after starting induction) produced by a differentiation method of Myoridge Co., Ltd. (protein-free differentiation method: see WO2015/182765) were thawed, and seeded into a 96 well plating (Corning) in a medium containing 2% FBS. The cells were seeded in 5 wells each at 1×10⁴ cells/well. After culturing the cells for 4 days, the medium was exchanged with serum-free DMEM (Gibco) medium. In a control well, AFP was not added, but AFP was added in the other wells in a concentration of 10 μg/mL or 1 μg/mL. After culturing the cells for another 7 days, the cells were immobilized with 50 μL of 4% paraformaldehyde (Nacalai), the resultant was washed once with 150 μL/well of a 0.01% Tween 20/PBS solution, and then, 200 nM phalloidin-FITC (Sigma) and 1 UM DAPI (Nacalai) were added thereto to stain actin filaments and cell nuclei. Thereafter, a fluorescence image of the phalloidin and DAPI of each well was taken with confocal quantitative imaging cytometer CQ1 (Yokogawa Electric Corporation) to calculate the amount of fluorescence per well.

### Results

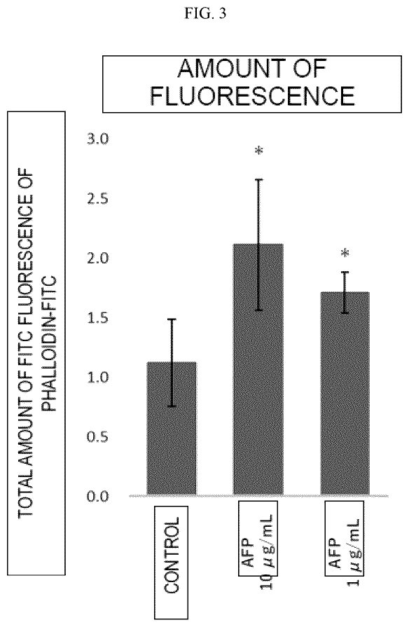

As compared with that in the control, there was no difference in the amount of fluorescence of DAPI in the wells to which AFP was added, but the amount of fluorescence of phalloidin was significantly increased (FIG. 3). Since phalloidin stains the actin filament, it is suggested that the cells had been matured to cardiomyocytes having developed cytoskeleton as compared with that in the control.

### Example 4

### 1. Influence on Cells of Various AFP Samples

It is known that an undifferentiated cell at an initial stage of development before differentiation is highly sensitive to toxicity as compared with a differentiated somatic cell, and therefore, various AFP samples were used to examine influence of AFP on the cell death before myocardial differentiation caused in Examples 1 and 2.

### Method

a. Buffer Replacement of AFP

AFP solutions of BioVision and LeeBio were respectively added to Amicon 10K columns, followed by centrifugation at 4° C. and 14,000×g for 20 minutes. A filtrate dropped at this point was collected, and used in an addition test (primary filtrate). Since each solution was concentrated to about 30 μl, 470 μl of serum-free DMEM medium (Gibco) was added thereto to a maximum volume of 500 μl, followed by centrifugation at 4° C. and 14,000×g for 20 minutes. This operation was repeated twice. To the resultant, 470 μl of serum-free DMEM medium (Gibco) was added again, and the resultant was lightly mixed, followed by collection by centrifugation with the column inverted. The centrifugation at this point was performed under conditions of 4° C., 14,000×g, and 2 minutes. The resultant was sterilized with a 0.22 μm filter, and then the addition test was conducted.

b. Addition to Cardiomyocyte

Frozen cardiomyocytes (Myoridge Co., Ltd., H-011106: 30 days after starting induction) produced by a differentiation method of Myoridge Co., Ltd. (protein-free differentiation method: see WO2015/182765) were thawed, and seeded into a 24 well plate (Corning). The number of cells was 1×10⁶ cells/well. After culturing the cells for 5 days, the cells were peeled off with Accumax (Nacalai), and seeded into a 96-well plate (Corning). The cells were seeded in 5 wells each at 8×10³ cells/well. After culturing the cells for 4 days, the medium was exchanged with serum-free DMEM medium (Gibco). Conditions were as follows.
1. Control: nothing added
2. HT-AFP: AFP (powder) manufactured by HyTest
3. Undiluted solution of BV-AFP: 1 mg/mL of AFP (liquid) manufactured by BioVision added to 10 μg/mL (ratio corresponding to 1%)
4. BV-AFP replacement: one obtained by buffer replacement
5. BV-AF filtrate: primary filtrate obtained in buffer replacement added to 1%
6. PBS: PBS added to 1%

After culturing the cells for another 5 days, the resultant cells were immobilized with 50 μL of 4% paraformaldehyde (Nacalai), the resultant was washed once with 150 ML/well of a 0.01% Tween 20/PBS solution, and then, 200 nM phalloidin-FITC (Sigma) was added thereto to stain actin filaments. The resultant was allowed to stand still overnight at 4° C., the entire amount of the staining solution was removed to be replaced with PBS, and a fluorescence image of the phalloidin of each well was taken with confocal quantitative imaging cytometer CQ1 (Yokogawa Electric Corporation) to calculate a phalloidin fluorescence area per well.

### Results

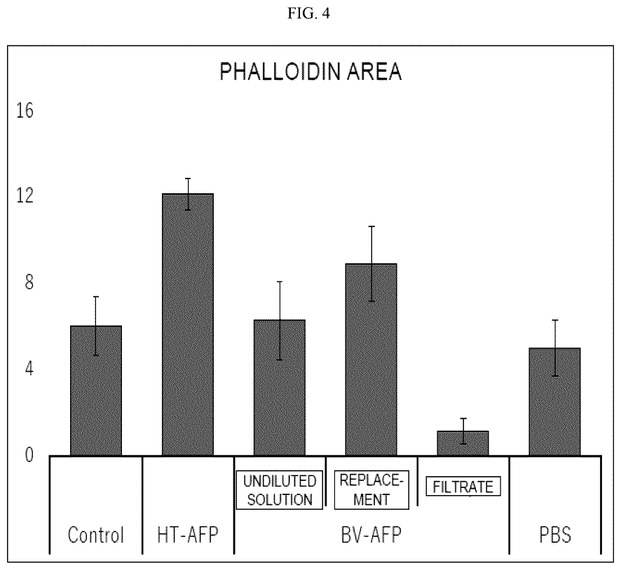

When the undiluted solution of BV-AFP was used, a phalloidin area value (indicating a cell area value) was substantially the same as that obtained under a condition where nothing was added, and thus the effect of the addition was not found, but in the case of replacement, the cell area value was increased to be close to a value of HT-AFP (FIG. 4). Besides, when the filtrate was added, cell death was caused. Based on these results, it is presumed that the buffer product is possibly highly cytotoxic to BV-AFP. On the other hand, even when the powder of HT-AFP was directly diluted and added, a higher value than that obtained under the condition where nothing was added was obtained, and hence, it is suggested to have substantially no toxicity (FIG. 4). In consideration of these results, HT-AFP was used to test the influence of the AFP addition on day 0 to day 6 after starting the differentiation again.

### 2. Influence on Myocardial Differentiation of AFP

Addition on Day 0 to day 6 after Starting Differentiation Method (Addition of 30 μg/mL AFP)

Human iPS cells (253G1 strain) were adherent cultured in Essential 8 in a 10 cm dish (Falcon, 353003) coated with iMatrix-511 (Matrixome). When 80 to 90% confluent, the cells were peeled off with EDTA, seeded at about 2×10⁶ cells/dish into 10 low adhesion 6 cm dishes (Corning, 3261), and cultured for 1 day to form an iPS cell spheroid. The thus obtained iPS cell spheroid was collected, and the whole amount thereof was seeded again uniformly in 21 wells of low adhesion 6-well plates (Corning, 3471).

A method of inducing differentiation into cardiomyocytes was conducted basically according to a method described in WO2015/182765 (see Example 1). In the same manner as in Example 1, differentiation of iPS cells was conducted under the following conditions A to D (n=3 for each). As a composition containing α-fetoprotein (AFP), human umbilical cord blood-derived AFP (HyTest, 8F8) was used.
(A) AFP not added (control)
(B) AFP added to a final concentration of 30 μg/mL in a period after starting differentiation from day 0 to day 2
(C) AFP added to a final concentration of 30 μg/mL in a period from day 3 to day 6
(D) AFP added to a final concentration of 30 μg/mL in a period from day 0 to day 6

The cells were collected on day 18, and from a half amount of the collected cells, RNA was collected with miRNeasy Mini Kit (Qiagen K.K.) to analyze expression levels of various genes by quantitative PCR (PowerUp SYBR Green Master Mix, Thermo Fisher Scientific) with TATA-Box Binding Protein (TBP) gene used as internal standard. The other half amount of the cells was subjected to single cell suspension by treatment with trypsin, and the number of cells was counted. Thereafter, a cardiomyocyte ratio (ratio of cTnT-positive cells) was checked by FACS analysis. In the FACS, Accuri C6 Plus (BD) was used.

### Results

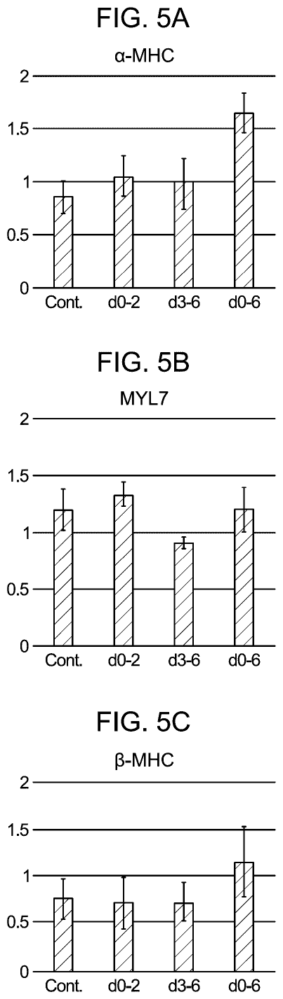

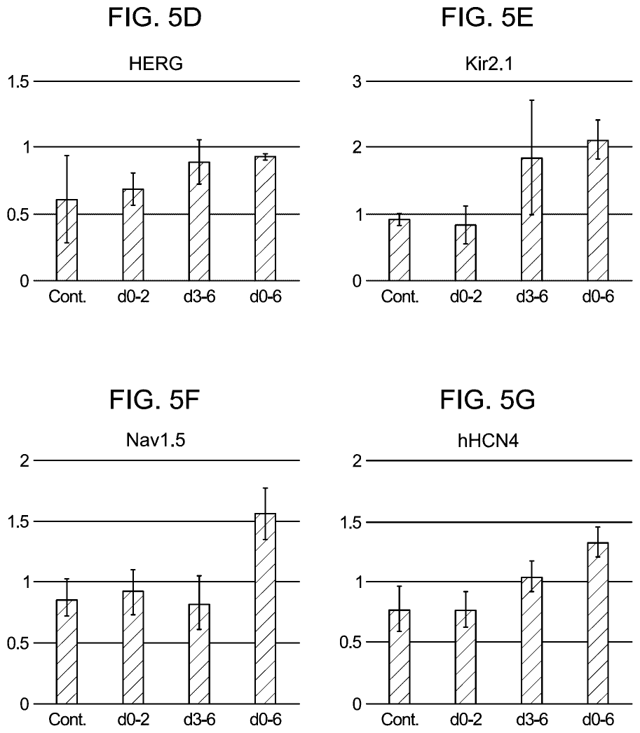

In the thus obtained cardiomyocytes, the number of cells was not largely changed as compared with that of the control under any of the conditions. On the other hand, a myocardial ratio was significantly increased under the conditions B to D. Besides, according to the analysis by quantitative PCR, the expression levels of several types of channel genes, and α- and β-MHC, and MYL7 were increased under the condition D, and it is thus suggested that the cells were cardiomyocytes developed as compared with those of the control and those obtained under the other AFP conditions (FIG. 5). As a result of comprehensive evaluation of the number of cells, the myocardial ratio, and the gene expression, it was found that the best result was obtained under the condition D. This reveals that continuous addition of AFP at least at an early stage of differentiation without limiting a period may be useful for differentiation and maturation of cardiomyocytes.

TABLE 4

| | | Number of Cells After | | | | cTnT |
|---|---|---|---|---|---|---|
| | | Single Cell Suspension | | | cTnT | Positive |
| | | Total | Live | Living Cell | Positive | Ratio |
| | | (×10e5) | (×10e5) | Ratio (%) | Ratio (%) | (NC, %) |
| A | A1 | 11.3 | 9.0 | 79.8 | 88.4 | 0.7 |
| AFP | A2 | 13.6 | 12.5 | 92.0 | 88.7 | 0.8 |
| not Added | A3 | 10.2 | 8.6 | 84.7 | 90.1 | 0.6 |
| B | C1 | 10.9 | 9.3 | 85.4 | 94.2 | 0.5 |
| day 0 to 2 | C2 | 13.7 | 12.3 | 89.5 | 90.7 | 0.3 |
| 30 μg/mL | C3 | 11.8 | 10.1 | 85.0 | 94.1 | 0.2 |
| C | E1 | 13.6 | 10.9 | 80.5 | 89.8 | 0.2 |
| day 3 to 6 | E2 | 11.1 | 9.2 | 82.7 | 83.4 | 0.0 |
| 30 μg/mL | E3 | 12.3 | 10.6 | 85.9 | 88.6 | 0.2 |
| D | G1 | 9.5 | 9.0 | 94.6 | 92.0 | 0.1 |
| day 0 to 6 | G2 | 9.2 | 8.6 | 94.4 | 92.3 | 0.1 |
| 30 μg/mL | G3 | 13.0 | 10.6 | 81.1 | 92.9 | 0.1 |

NC: Negative Control

### Example 5

Method (Addition of 30 μg/mL AFP)

Frozen cardiomyocytes (Myoridge Co., Ltd., H-011106: 30 days after starting induction) produced by a differentiation method of Myoridge Co., Ltd. (protein-free differentiation method: see WO2015/182765) were thawed, and seeded into a 96-well plate (Corning) in a serum-free medium. The cells were seeded in 5 wells each at 2×10⁴ cells/well. After culturing the cells for 8 days, the medium was exchanged with serum-free DMEM medium (Gibco). As a composition containing α-fetoprotein (AFP), human umbilical cord blood-derived AFP (HyTest, 8F8) was used. Conditions were as follows.
(A) nothing added (control)
(B) 2% FBS
(C) 30 μg/mL HSA
(D) 30 μg/mL AFP (derived from umbilical cord blood)
(E) 30 μg/mL AFP (HEK recombinant)
(F) 15 μg/mL AFP (derived from umbilical cord blood) and 15 μg/mL of HSA

After culturing the cells for another 7 days, the resultant cells were immobilized with 50 ML of 4% paraformaldehyde (Nacalai), the resultant was washed once with 150 ML/well of a 0.01% Tween 20/PBS solution, and then, 200 nM phalloidin-FITC (Sigma) and 400 ng/ml cTnT primary antibody (Santa Cruz), and 1 μM DAPI (Nacalai) were added thereto to stain actin filaments and cell nuclei. The resultant was allowed to stand still overnight at 4° C., the entire amount of the staining solution was removed, and cTnT secondary antibody, Alexa Flour 647 (Thermo Fisher) was added thereto, followed by a reaction caused at room temperature for 30 minutes to stain cardiac troponin T. Thereafter, the resultant was replaced with PBS, and fluorescence images of the phalloidin, cTnT, and DAPI of each well were taken with confocal quantitative imaging cytometer CQ1 (Yokogawa Electric Corporation) to calculate the amounts of phalloidin and cTnT fluorescence and the number of DAPI per well.

### Results

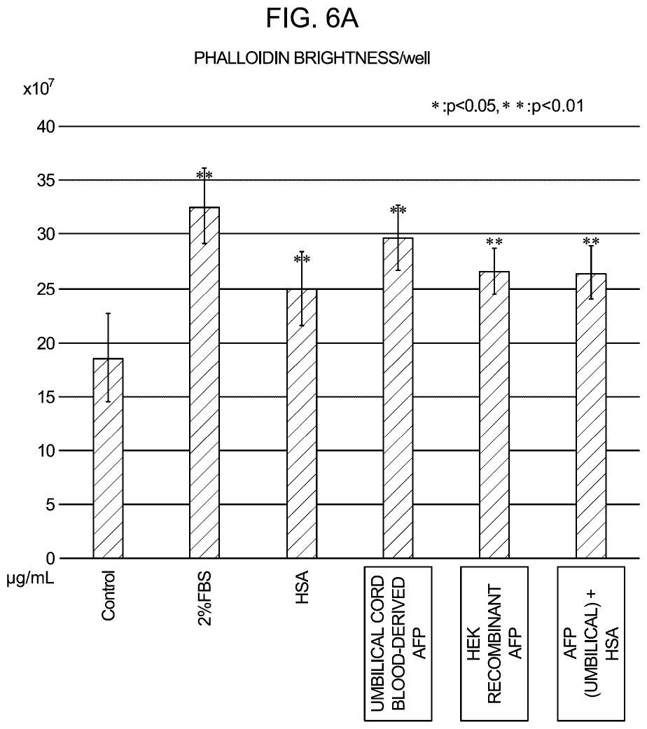

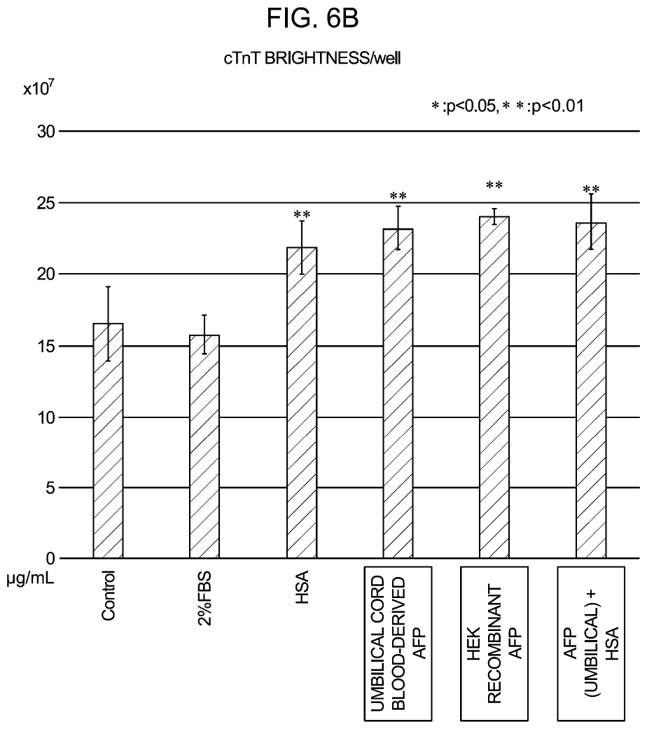

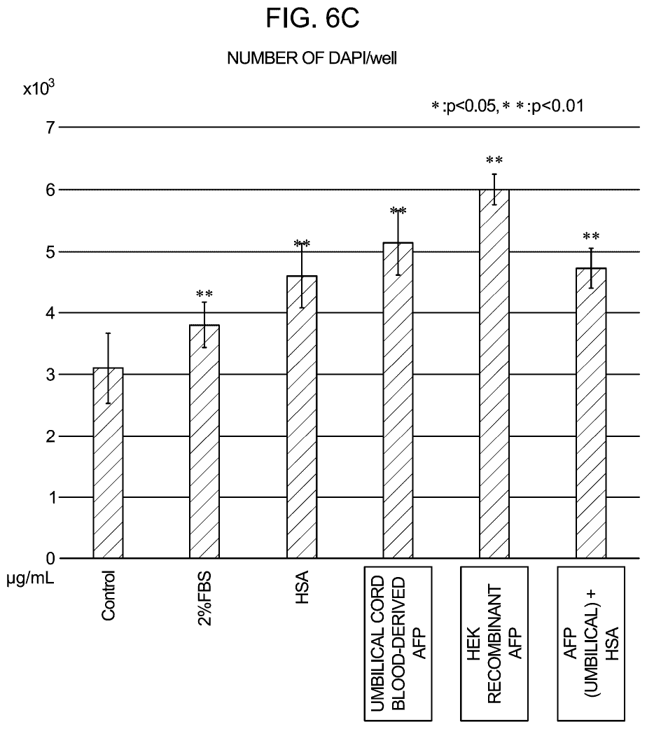

As compared with the control and the condition of adding FBS, the amount of cTnT fluorescence and the number of DAPI were significantly increased in the wells to which AFP was added (FIG. 6). This suggests that the cardiomyocytes obtained by adding AFP have developed myocardial fibers and are matured cells. Besides, the number of cells was also large, and an effect of causing cardiomyocytes to survive was also exhibited.

### INDUSTRIAL APPLICABILITY

The present invention is useful for preparing cardiomyocytes for regenerative medicine.

1. A method for producing a cell population comprising cardiomyocytes, comprising differentiation process from cells capable of differentiating into cardiomyocytes to cardiomyocytes, wherein the differentiation process comprises culturing the cells in a serum-free medium containing α fetoprotein to obtain the cardiomyocytes.

2. The method according to claim 1, wherein the cells capable of differentiating into cardiomyocytes are pluripotent stem cells or mesodermal cells derived from pluripotent stem cells.

3. The method according to claim 2, wherein the pluripotent stem cells are iPS cells.

4. The method according to claim 1, wherein the obtained cardiomyocytes are cardiomyocytes having a high expression level of hERG, KCNJ2, SCN5A, or MYL7.

5. The method according to claim 1, wherein the obtained cardiomyocytes are mature cardiomyocytes.

6. The method according to claim 1, wherein the cells are cultured in the serum-free medium containing α fetoprotein from start of the differentiation until beating of the cells is confirmed.

7. The method according to claim 1, wherein the cells are cultured in the serum-free medium containing α fetoprotein after beating of the cells is confirmed.

8. The method according to claim 1, wherein the obtained cell population comprises cardiomyocytes in a ratio of 80% or more when measured by flow cytometry.

9. The method according to claim 1, wherein the α fetoprotein is human α fetoprotein.

10. The method according to claim 1, wherein the serum-free medium contains the α fetoprotein in an amount of 5 to 100 g/mL.

11. The method according to claim 1, wherein the serum-free medium is xeno-free and/or cytokine-free.

12. The method according to claim 1, wherein the serum-free medium is protein-free excluding α fetoprotein.

13. A serum-free medium, comprising α fetoprotein, and a differentiation inducing factor for a cardiomyocyte.

14. The medium according to claim 13, wherein the serum-free medium is xeno-free and/or cytokine-free.

15. The medium according to claim 13, wherein the serum-free medium is protein-free excluding α fetoprotein.

16. A method for promoting cardiomyocyte maturation, comprising contacting cells capable of differentiating into cardiomyocytes to α fetoprotein.

17. A culture of a cell population comprising cardiomyocytes, wherein the culture comprises α fetoprotein but does not comprise serum.
